(19) United States
(12) Plant Patent Application Publication
(10) Pub. No.: US 2025/0008854 P1
(43) Pub. Date: Jan. 2, 2025

(54) MANDARIN PLANT NAMED 'RES 19-56'

(50) ;

(71) Applicant: Florida Foundation Seed Producers, Inc., Marianna, FL (US)

(72) Inventor: Frederick GMITTER, Jr., Lakeland, FL (US)

(21) Appl. No.: 18/751,578

(22) Filed: Jun. 24, 2024

Related U.S. Application Data
(60) Provisional application No. 63/510,556, filed on Jun. 27, 2023.

(51) Int. Cl. A01H 6/78

(52) U.S. Cl. CPC A01H 6/785
USPC PLT/202

(57) ABSTRACT

A new and distinct seedless, easy to peel mandarin hybrid, named ‘RES 19-56’, with a unique clustered fruit bearing habit that can appeal to citrus hobbyists as an ornamental, but that also has shown potential value for premium early season fresh market production by CUPS or other citrus growers. Has shown the ability to set large numbers of parthenocarpic fruit in the CUPS production system. These clusters sometimes number 15 or more fruit, having many more than fruit per cluster than other seedless mandarin hybrids.

### CROSS-REFERENCE TO RELATED APPLICATIONS

This Application claims the benefit of U.S. Provisional Patent Application No. 63/510,556, filed Jun. 27, 2023, which is incorporate by reference herein in its entirety.

### ACKNOWLEDGEMENT OF FEDERAL RESEARCH SUPPORT

N/A.

Genus and species: Citrus reticulata hybrid.

Cultivar denomination: ‘RES 19-56’.

### BACKGROUND OF THE NEW CULTIVAR

The present invention relates to a new and distinct cultivar of citrus (mandarin), botanically known as Citrus reticulata and hereinafter referred to by the name ‘RES 19-56’.

The new mandarin cultivar ‘RES 19-56’ arose from a cross of ‘Daisy’ mandarin hybrid (not patented) x ‘Mukaku Kishu’ (also known as ‘Seedless Kishu’ mandarin; not patented), both publicly available and unprotected cultivars. The cross was made in 2005 in the experimental orchard in Gainesville, Florida. Seeds from the cross were planted in the greenhouse, and then following germination and seedling growth, a bud was grafted to ‘Carrizo’ citrange rootstock (not patented). The young, grafted tree was then grown off as a single stem plant with no branching. The tree was planted with many other hybrids with 0.3 m distance between each in the row. They were continuously trained to a single stem by repeated removal of all side sprouts as they appeared, and supported by a wire trellis until they reached approximately 4 m in height. At that time, the tops were bent over to induce spring flowering and fruit set. The new cultivar ‘RES 19-56’ was selected in 2010 and again in 2011 and entered into the Florida Department of Plant Industry (DPI) Parent Tree Program for cleanup, indexing, and certification in 2014. Upon certification in 2016, several trees were propagated and planted in the field in April 2017 in Pasco County, FL. Fruit were first observed on these trees in 2019 and were found to be true to type. Additional trees were propagated and planted into a commercial citrus under protective screen (CUPS) structure in 2019, and first crops were observed in 2021, which also produced true-to-type fruit.

Plant Breeder's Rights for this cultivar have not been applied for. The new citrus (mandarin) cultivar ‘RES 19-56’ has not been made publicly available more than one year prior to the priority filing date of this application.

### SUMMARY OF THE INVENTION

The new mandarin cultivar ‘RES 19-56’ has not been observed under all possible environmental conditions. The phenotype of the new cultivar may vary with variations in environment and cultural practices such as temperature, light intensity, fertilization, irrigation, and application of plant growth regulators without any change in genotype.

Trees of the new cultivar ‘RES 19-56’ are compact and bear fruit precociously, beginning in the second year after planting. This cultivar has a unique ability to set clusters of fruit that sometimes number as many as 15 fruit, or more. The fruit are completely seedless, very easy to peel, color well in the early season (September-November), and have good flavor. Individual fruit tend to have a neck at the stem end. It appears that they can set parthenocarpic fruit in the CUPS production system. By contrast, some other seedless hybrids in CUPS trials have not set many fruits, despite flowering normally.

Juice quality measurements for the new cultivar ‘RES 19-56’ were taken in an early harvest of 15 Sep. 2021: Brix was 9.9 and acidity was 0.6, for a ratio of 16.5, which is indicative of the very early maturity with good fruit quality. It was reported that fruit harvested at that time were green but developed good color following ethylene treatment and held up well in storage with no decay.

The trees grown in the field in Pasco County, Florida have held up well against citrus huanglongbing or greening (HLB). Production has been good, with trees also exhibiting precocity and the fruit clustering phenotype as well. Experiments in CUPS systems have been closely monitored, and good potential for the fresh market production of early maturing mandarin fruit by ‘RES 19-56’ has been seen. Trees of ‘RES 19-56’ appear to be more tolerant of certain pests and diseases. To date, no major disease problems have been noted, though systematic testing has not taken place.

While the fresh citrus market may be an important destination of fruit from ‘RES 19-56’, the unique and attractive appearance of the trees and particularly the many large clusters of brightly colored and parthenocarpic fruit provide another interesting possibility for commercialization. With its compact tree structure and obvious ornamental value, this cultivar could be an important addition to the inventories of nurseries who cater to this market.

### DESCRIPTION OF THE FIGURES

The accompanying photographs (as shown in FIGS. 1-3) illustrate the overall appearance of the new mandarin cultivar ‘RES 19-56’. These photographs show the colors as true as can be reasonably obtained in colored reproductions of this type. Colors in the photographs may differ slightly from the color values cited in the detailed botanical description, which accurately describe the colors of the new mandarin cultivar.

FIG. 1A shows a four-year-old tree of the new mandarin cultivar ‘RES 19-56’ grown on ‘US 897’ rootstock in a CUPS structure, showing tree shape and spreading habit;

FIG. 1B shows a graft union between the new cultivar ‘RES 19-56’ and ‘US 897’ rootstock, as well as internal branching habit, in a CUPS structure;

FIG. 1C shows a field-grown tree of the new cultivar with mature fruit in early November 2023 in north central Florida;

FIG. 2A shows clusters of fruit set on a two-year-old tree of the new cultivar at fruit maturity in a CUPS structure in October 2021 (planted in 2019);

FIG. 2B shows clusters of fruit set on another two-year-old tree of the new cultivar at fruit maturity in a CUPS structure in October 2021 (planted in 2019);

FIG. 2C shows a large cluster of fruit growing on one stem of a tree of the new cultivar in a CUPS structure in October 2021;

FIG. 3A shows fruit from a field-grown tree of the new cultivar in north central Florida, harvested in early November 2023, with easily removed peel, intact segments, spherical shape, and slight neck at the stem end of the fruit;

FIG. 3B shows fruit harvested from trees of the new cultivar grown in a CUPS structure;

FIG. 3C shows juice samples from the individual fruit shown in

FIG. 3B;

FIG. 3D shows fruit harvested in late October 2023 from trees of the new cultivar grown in a CUPS structure, cut equatorially;

FIG. 3E shows a close-up view of whole and cut mature fruit of the new cultivar, harvested mid-November 2023 from trees grown in a CUPS structure;

FIG. 4A shows flowering of young flush on a tree of the new cultivar grown in a CUPS structure in Spring 2023;

FIG. 4B shows a close-up view of flowers on a tree of the new cultivar grown in a CUPS structure in Spring 2023, showing range from pre-anthesis through post-anthesis, including one small green fruitlet;

FIG. 4C shows a close-up view of buds and flowers alongside a rule for measurement;

FIG. 4D shows a close-up view of buds and flowers alongside a rule for measurement;

FIG. 4E shows a close-up view of buds and flowers alongside a rule for measurement;

FIG. 5A shows a close-up view of leaves of the new cultivar alongside a ruler for measurement;

FIG. 5B shows a close-up view of leaves of the new cultivar alongside a ruler for measurement; and

FIG. 6 shows typical bud sticks cut for propagation from the new cultivar.

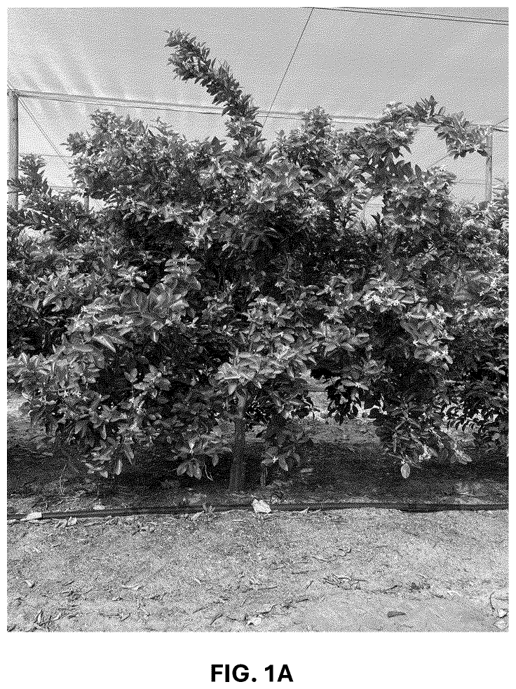

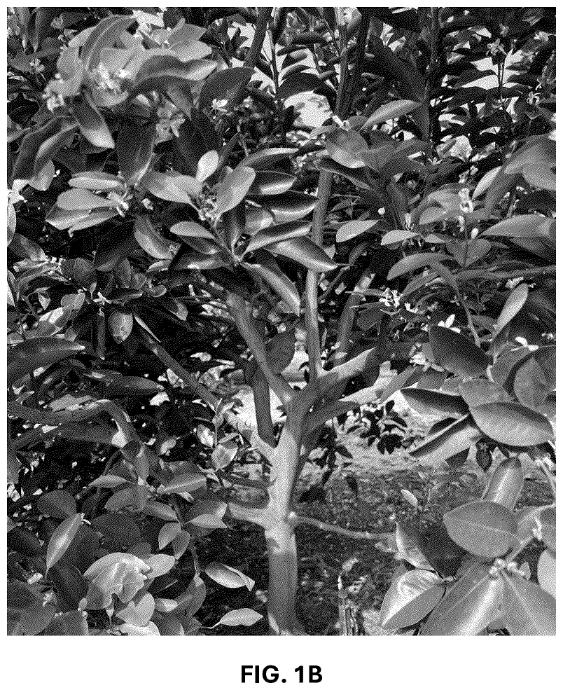

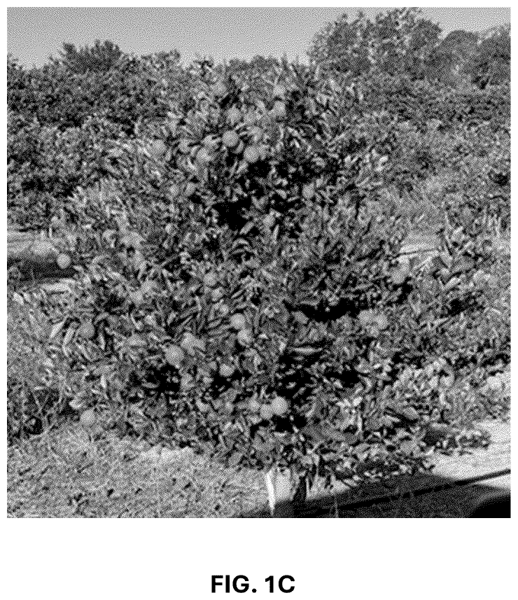

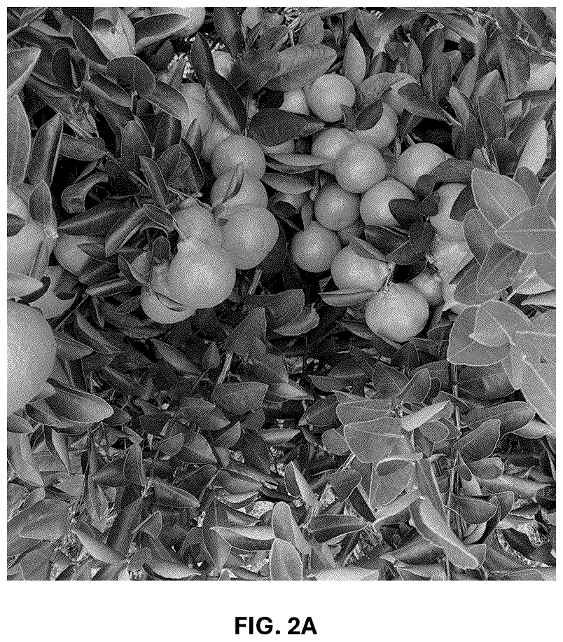

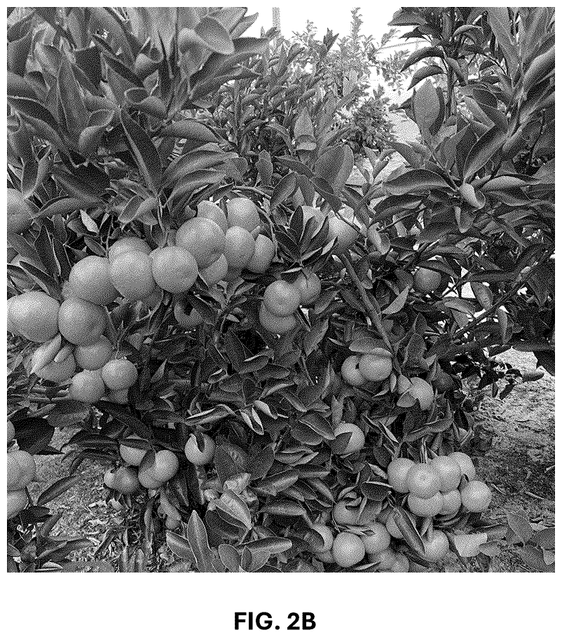

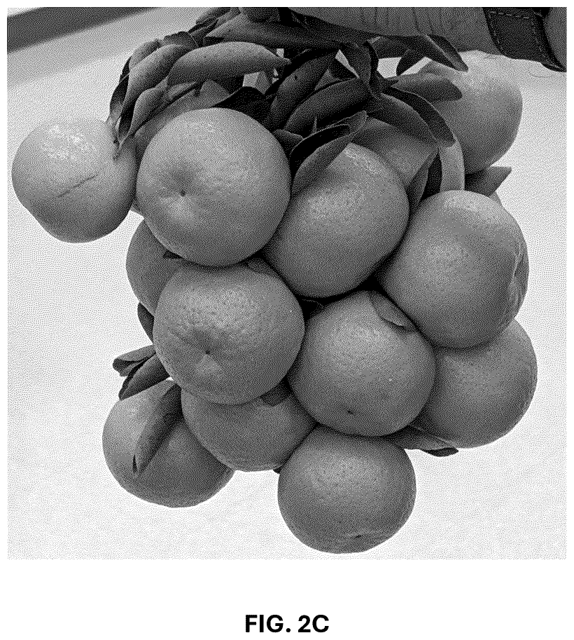

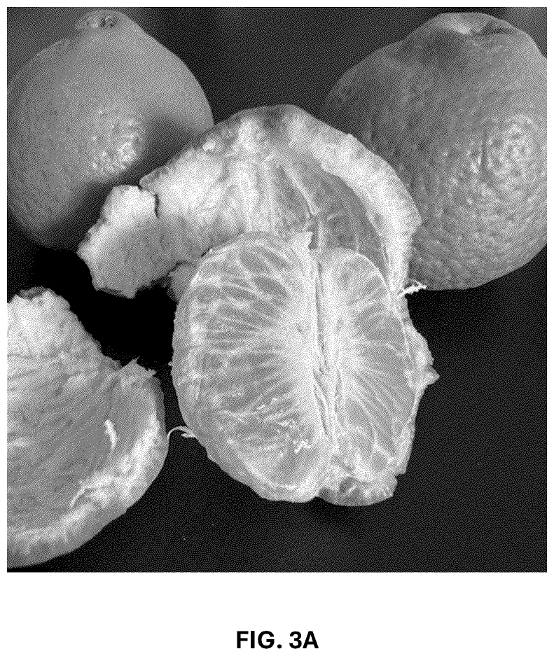

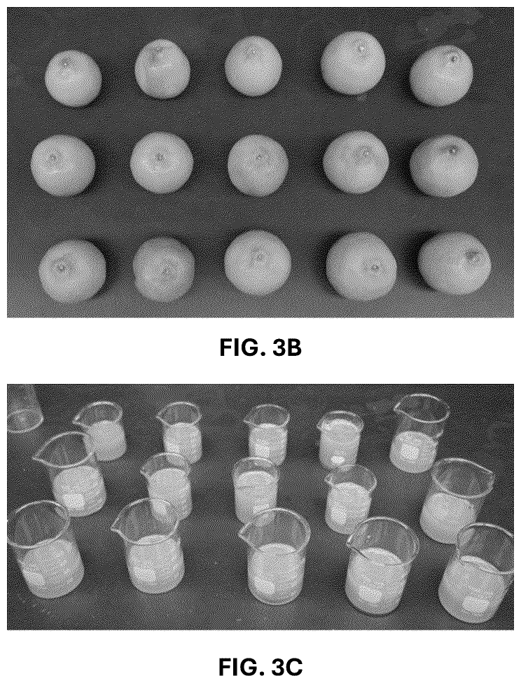

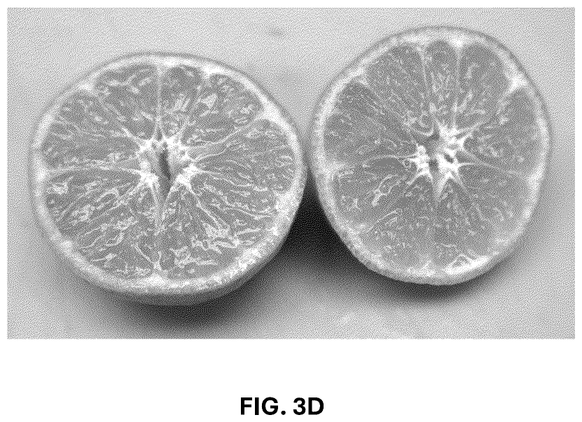

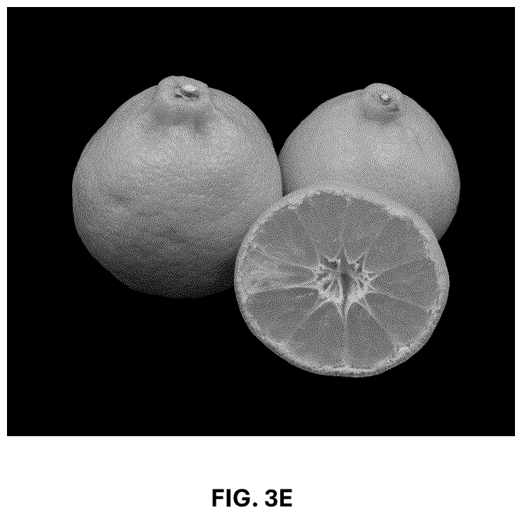

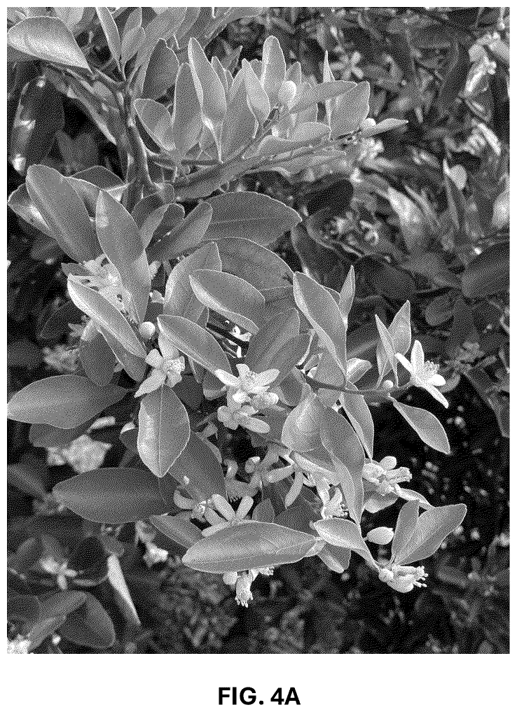

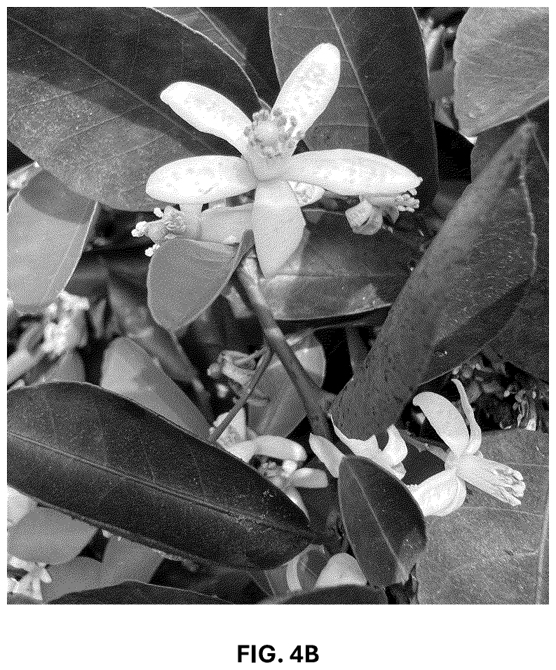

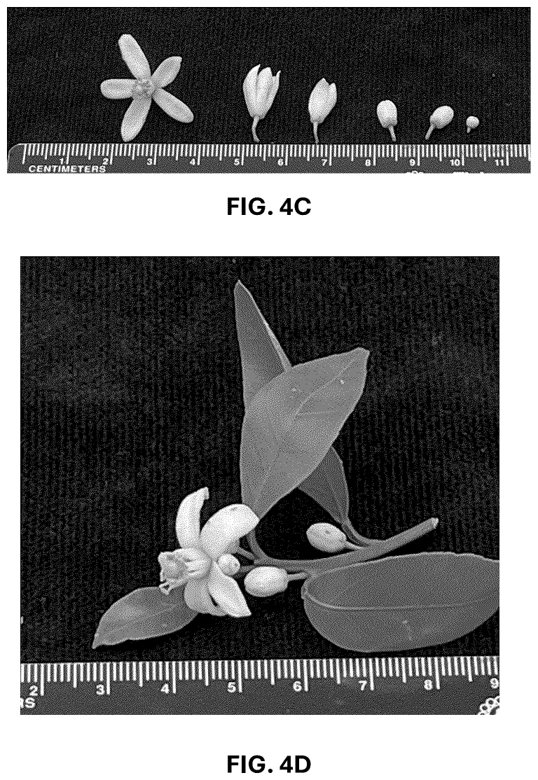

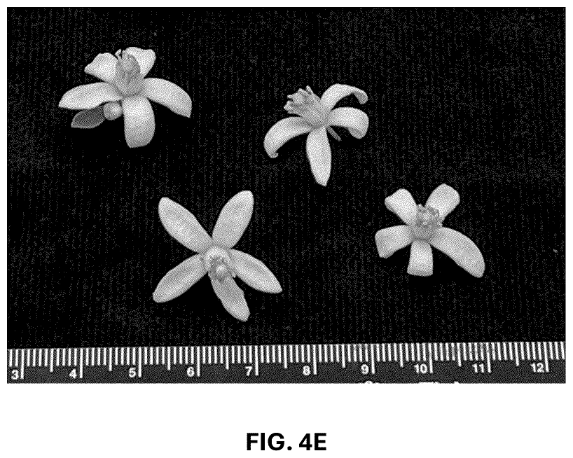

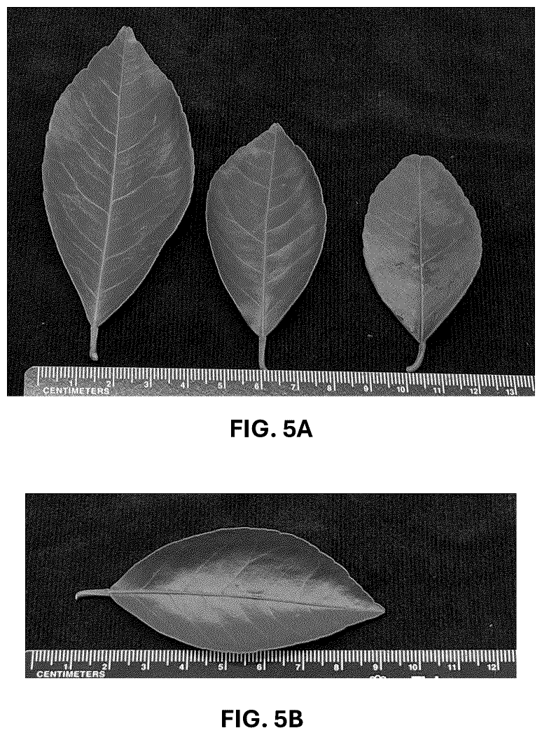

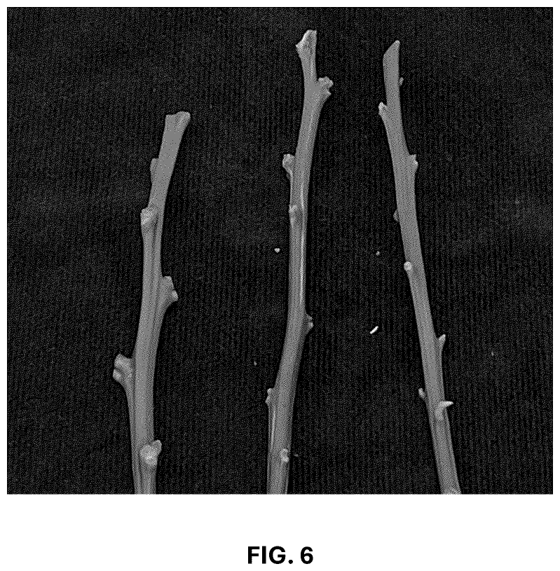

### DETAILED BOTANICAL DESCRIPTION OF THE CULTIVAR

In the following description, color references are made to The Royal Horticultural Society (R.H.S.) Colour Chart, 1986 Edition, except where general terms of ordinary dictionary significance are used.

Vegetative descriptions are based on 4.5-year-old trees on ‘US 897’ rootstock (not patented), grown in a covered structure (CUPS) in Central Florida, and free of HLB.
Botanical description:
   Botanical classification.—Family: Rutaceae. Botanical name: Citrus reticulata hybrid. Common name: Mandarin. Cultivar: ‘RES 19-56’.
   Parentage.—‘Daisy’ mandarin x ‘Mukaku Kishu’ (also known as ‘Seedless Kishu’), both publicly available and unprotected cultivars.
   Tree.—Ploidy: Diploid. Size: Medium. Width across row: 2.3 meters on average. Width down row: 2.4 meters on average. Tree height: 2.6 meters on average. Tree canopy diameter: 2.7 meters on average. Vigor: Moderate. Density: Dense foliage. Tree shape (form): Oblate (obloid) to round, spreading. Growth habit (current season): Vegetative growth in general is moderate.
   Trunk.—Trunk diameter: Approximately 6.2 cm at 30 cm above the ground. Trunk texture: Relatively smooth. Trunk bark color: RHS N199A.
   Branches.—Branch length: 2 meters on average. Branch diameter: 3.8 cm on average. Crotch angle: Acute with a degree of 75-80 on average (less than 90 degrees) in the middle of the tree, with some on the outside being more than 90 degrees (110-115). Branch texture: Relatively smooth with very small spines. Branch color: RHS 199B (Grey-Brown Group).
   Leaves.—Size (lamina average): Length: 111 mm on average. Width: 45 mm on average. L/W ratio: 2.4 on average. Thickness: Regular (average thickness). Type: Simple. Shape: Elliptical. Apex: Acute to retuse. Base: Acute. Margin: Dentated with split end. Surface: Upper surface: Smooth. Lower surface: Medium veins that are pinnately netted. Color: Upper surface (adaxial): RHS 137A (Green Group). Lower surface (abaxial): RHS 137D (Green Group). Petiole: Shape and attachment: Brevipetiolate; shorter than the leaf lamina. The junction between the petiole and the lamina is articulate. Shape (petiole wing): Obovate with a narrow end at the base. Length: 11 mm on average. Width: 1-1.1 mm on average. Color: RHS 137B (Green Group).
   Flowers.—Type: Perfect. Flowers bearing: Single or clusters. Branches have flowers grown in clusters. Each cluster consists of 3-5 flowers. Individual flowers are grown from leafy terminals and leaf axillaries. Flower diameter: 20 mm on average. Flower depth: 17 mm on average. Flower blooming period: February to March, depending on season. First bloom: Earliest is generally the 1^{st} of February. Full bloom: Late February to early March, depending on season. Fragrance: Fragrant. Flower buds' size: Length: 1.5 mm on average for the initial visible flower bud; 2.3 mm on average for the mature flower buds before opened. Diameter: 1.2 mm on average in the initial visible flower bud; 5.2 mm on average in the mature bud. Flower bud shape: Initial visible flower bud with round dome shape; mature flower bud with elongated olive shape. Flower bud color: RHS 144C (yellow-green) for the initial visible flower bud; RHS NN 155B (white) for mature buds.
   Reproductive organs.—Fertility: Pollen fertile, female sterile. Completely seedless by virtue of arrested embryo development, which is typical of ‘Mukaku Kishu’ F1 hybrids. Petals: Number: 5. Petal length: 14 mm on average. Petal width: 4.7 mm on average. Petal shape: Flat spatula shape. Apex shape: Smooth with obtuse angle. Base shape: Obtuse. Petal Color: Upper Surface: RHS NN 155C (White Group). Lower surface: RHS NN 155B (White Group). Margin: Smooth. Sepal: Number (per flower): 5. Shape: Delta shaped with an acute angle at apex, although some with flat angle. Length: 3.5 mm on average. Width: 2 mm on average. Apex shape: Acute angle at apex. Margin: Smooth. Color: Upper surface: RHS 158A (Green-White Group). Lower surface: RHS 157A (Green-White Group). Pedicel: Length: 8 mm on average. Diameter: 1.5 mm on average. Color: RHS 144A (Yellow-Green Group). Stamen: Number: 23 per flower on average. Length: 14 mm on average. Anther: Length: 2 mm on average. Width: 0.7 mm on average. Color: RHS 14B (Yellow Orange Group). Pollen color (general): RHS 13A (Yellow Group). Pistil: Number: 1. Length: 14 mm on average. Color: RHS 153B (Yellow Green Group). Style length: 12 mm on average. Style diameter: 1.4 mm on average. Style color: RHS 144A (Yellow Green Group). Ovary shape: Oval shape. Ovary diameter: 4 mm on average. Ovary color: RHS 145C (Yellow Green Group).
   Fruit.—Size: Small. Average weight (per individual fruit): 62.7 g on average. Length (height): 6.5 cm on average. Diameter: 6.35 cm on average. Shape: Spherical. Shape (cross-section): Round. Apex: Round with a small or slight neck at the stem end of the fruit. Base: Round. Harvest: Date of first pick: 15 September. Date of last pick: 15 November. Fruit stem: Length: 10 mm on average. Diameter: 2 mm on average. Color: RHS 189A (Greyed-green Group).
   Rind.—Adherence: Easy to peel. Thickness: Thin (1.2 mm on average). Texture: Medium firm. Surface Texture: Medium smooth. Color: Flavedo (epicarp): RHS N25B (Orange Group). Albedo (mesocarp): RHS N19D (Yellow-Orange Group). Style end: Closed. Rind oil cell density: 70-100 cell/square cm. Oil gland size: 0.5 mm on average.
   Flesh.—Number of segments: 10 on average. Segment length: Not available. Segment width: 1.5 cm. Juice: Abundant. Color: RHS 23B Yellow-Orange Group. Texture: Soft to medium. Vesicle thickness: Thin, tender. Length: 5 mm on average. Diameter: 2 mm on average. Juice quality (from early harvest on 15 Sep. 2021): Brix: 9.9.Acidity (average): 0.6%. Juice color: Not determined. Lb solid/box: Not determined, fresh market variety, not intended for juice processing. Juice quality (from mid-season harvest on 24 Oct. 2022): Brix: 10.4. Acidity (average): 0.43%. Ratio: Approximately 24. Juice color: RHS 23B (Yellow-Orange Group). Lb solid/box: Not determined, fresh market variety, not intended for juice processing.
   Seeds.—Presence: Completely seedless, no seeds ever observed. Fruit parthenocarpy: Yes, fruit set in protected structure in the absence of pollinating insects and without limb girdling or applications of gibberellic acid to support fruit set.
   Resistance to disease.—No systematic disease resistance testing conducted. No obvious disease problems noted, very clean fruit produced with minimal chemical treatments to control diseases.

What is claimed is:

1. A new and distinct mandarin plant named ‘RES 19-56’ as illustrated and described herein.
